(19) United States
(12) Patent Application Publication
(10) Pub. No.: US 2026/0279754 A1
(43) Pub. Date: Sep. 17, 2026

(54) SYSTEMS AND METHODS FOR MULTI-CHANNEL MASS SPECTROMETRY

(71) Applicant: Peninsula Technologies, LLC, Wilmington, DE (US)

(72) Inventor: Viatcheslav V. KOVTOUN, Wilmington, DE (US)

(21) Appl. No.: 19/682,258

(22) Filed: May 19, 2026

Related U.S. Application Data
(63) Continuation of application No. 19/652,878, filed on Apr. 20, 2026.
Application No. 19/652,878 is a continuation of application No. PCT/US2024/051812, filed on Oct. 17, 2024.
(60) Provisional application No. 63/592,127, filed on Oct. 20, 2023.

(51) Int. Cl. H01J 49/00; G01N 27/623; G01N 33/483; H01J 49/06; H01J 49/16; H01J 49/40

(52) U.S. Cl. CPC H01J 49/009; G01N 27/623; G01N 33/483; H01J 49/005; H01J 49/063; H01J 49/165; H01J 49/40

(57) ABSTRACT

Described herein are multichannel mass spectrometry devices and methods of using said devices. In some cases, the devices and methods include analyzing overlapping mass windows.

### CROSS REFERENCE

This application is a continuation of U.S. patent application Ser. No. 19/652,878, filed Apr. 20, 2026, which is a continuation of International Patent Application No. PCT/US2024/051812 filed Oct. 17, 2024, which claims the benefit of U.S. Provisional No. 63/592,127, filed on Oct. 20, 2023, each of which are incorporated herein by reference in their entirety.

### BACKGROUND

Over the past few decades, mass spectrometers and analysis methods based on mass spectrometry have seen substantial improvements in instrument sensitivity and resolution, which has expanded their application into nearly every branch of physical science. There are, however, problems in these fields that remain unaddressed. For example, mass spectrometers, particularly those with high mass resolving powers, can suffer from substantial losses of ion flux before reaching a mass analyzer, which can diminish the detection and/or quantification of analytes. Accordingly, improved mass spectrometers and mass spectrometry-based methods of analysis are needed to mitigate these losses.

### SUMMARY

Provided herein, in some embodiments, is an apparatus for parallel mass spectrometry analysis comprising: one or more ion mobility devices configured to receive ions from an ion source; a first mass filter configured to receive mobility-separated ions from the one or more ion mobility devices; a second mass filter configured to receive mobility-separated ions from the one or more ion mobility devices; a first collision cell configured to receive mass-filtered ions from the first mass filter; a second collision cell configured to receive mass-filtered ions from the second mass filter; a first mass analyzer configured to analyze ions received from the first collision cell; and a second mass analyzer configured to analyze ions received from the second collision cell.

Provided herein, in some embodiments, the apparatus further comprises a third mass filter configured to receive mobility-separated ions from the one or more ion mobility devices; a third collision cell configured to receive mass-filtered ions from the third mass filter; and a third mass analyzer configured to analyze ions received from the third collision cell. In some embodiments, the apparatus further comprises a fourth mass filter configured to receive mobility-separated ions from the one or more ion mobility devices; a fourth collision cell configured to receive mass-filtered ions from the fourth mass filter; and a fourth mass analyzer configured to analyze ions received from the fourth collision cell. In some embodiments, the first mass filter is configured to select first mass windows, wherein the second mass filter is configured to select second mass windows, wherein the first mass windows and the second mass windows are different, and wherein the first mass windows and the second mass windows overlap.

Provided herein, in some embodiments, is a method for parallel mass spectrometry analysis comprising: introducing gas-phase ions obtained from a sample into one or more ion mobility devices; sequentially introducing windows of ion mobilities from the one or more ion mobility devices to a first mass filter; sequentially introducing windows of ion mobilities from the one or more ion mobility devices to a second mass filter; sequentially introducing windows of ion masses from the first mass filter to a first collision cell; sequentially introducing windows of ion masses from the second mass filter to a second collision cell; sequentially analyzing windows of ions received from the first collision cell using a first mass analyzer; and sequentially analyzing windows of ions received from the second collision cell using a second mass analyzer. In some embodiments, the one or more ion mobility devices comprise a first ion mobility device and a second ion mobility device, and wherein the method comprises: introducing a first portion of the gas-phase ions into the first ion mobility device; introducing a second portion of the gas-phase ions into the second ion mobility device; sequentially introducing windows of ion mobilities from the first ion mobility device to the first mass filter; and sequentially introducing windows of ion mobilities from the second ion mobility device to the second mass filter. In some embodiments, the ion source comprises a first electrospray ionization device and second electrospray ionization, wherein the method comprises: introducing gas-phase ions from the first electrospray ionization device to the first ion mobility device; and introducing gas-phase ions from the second electrospray ionization device to the second ion mobility device. In some embodiments, the method comprises: sequentially introducing windows of ion mobilities from the one or more ion mobility devices to a third mass filter; sequentially introducing windows of ion masses from the third mass filter to a third collision cell; and sequentially analyzing windows of ions received from the third collision cell using a third mass analyzer. In some embodiments, the method comprises: sequentially introducing windows of ion mobilities from the one or more ion mobility devices to a third mass filter; and sequentially analyzing windows of unfragmented ions received from the mass filter using a third mass analyzer. In some embodiments, the method further comprises: sequentially introducing windows of ion mobilities from the one or more ion mobility devices to a fourth mass filter; sequentially introducing windows of ion masses from the fourth mass filter to a fourth collision cell; and sequentially analyzing windows of ions received from the fourth collision cell using a fourth mass analyzer.

Provided herein in some embodiments, wherein the first mass filter selects first mass windows, wherein the second mass filter selects second mass windows, wherein the first mass windows and second mass windows are different, and wherein the first mass windows and the second mass windows overlap. In some embodiments, wherein pairs of mass windows that overlap are both analyzed within less than 1 second, less than 500 milliseconds, less than 50 milliseconds, less than 25 milliseconds, less than 15 milliseconds, less than 10 milliseconds, or less than 5 milliseconds.

Provided herein in some embodiments, is a parallel mass analyzer comprising: a flight tube; an ion mirror; one or more ion detectors; one or more ion optics elements; an accelerator configured to eject first ions and second ions into the flight tube, wherein the first ions are directed by the ion mirror to a first region of the one or more detectors, and wherein the second ions are directed by the ion mirror to a second region of the one or more ion detectors. In some embodiments, the first ions and the seconds ions are separately detected by the one or more detectors. In some embodiments, the ion detector comprises: a microchannel plate; a first anode operably coupled to the microchannel plate and configured to receive the first ions directed to the first region of the ion detector; and a second anode operably coupled to the microchannel plate and configured to receive the second ions directed to the second region of the ion detector. In some embodiments, wherein the one or more ion detectors comprise: a first ion detector configured to receive the first ions directed to the first region; and a second ion detector configured to receive the second ions directed to the second region. In some embodiments, the parallel mass analyzer further comprises a second flight tube, a second ion mirror and a second one or more detectors, wherein the accelerator is configured to eject third ions and fourths ions into the second flight tube, wherein the third ions are directed by the second ion mirror to a first region of the second one or more detectors, and wherein the fourth ions are directed by the second ion mirror to a second region of the second one or more ion detectors.

Provided herein, in some embodiments, is a method for parallel mass analysis using the mass analyzer, the method comprising: injecting the first ions into the flight tube using the accelerator; injecting the second ions into the flight tube using the accelerator; directing the first ions to the first region of the one or more ion detectors using the ion mirror; directing the second ions to the second region of the one or more ion detectors using the ion mirror; determining m/z for at least a portion of the first ions; and determining m/z for at least a portion of the second ions.

Provided herein, in some embodiments, is a method of identifying analytes comprising: obtaining data representing signal intensity against m/z for a plurality of overlapping mass windows; comparing the overlapping mass windows to associate peaks with a narrower mass range that overlaps between two or more mass windows; and identifying one or more analytes based on the narrower mass range and the peaks associated with the narrower mass range.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 illustrates an example of a dual-channel configuration for a mass spectrometry device that is within the scope of the present application.

FIG. 2 illustrates an example of a block diagram for a dual-channel configuration for a mass spectrometry device that is within the scope of the present application.

FIG. 3 illustrates an example of using overlapping mass windows to analyze a sample using mass spectrometry that is within the scope of the present application.

FIG. 4 illustrates an example of different scenarios for analyzing a sample by mass spectrometry by using (i) narrow mass windows in a single channel, (ii) wider, overlapping windows in a single channel, and (iii) wider, overlapping windows in a dual channel.

FIG. 5 illustrates an example of a schematic for a dual time-of-flight mass analyzer that shares a common flight tube, accelerator, and reflectron that is within the scope of the present application.

FIG. 6 illustrates an example of a schematic for a dual time-of-flight mass analyzer that shares a common flight tube, accelerator, reflectron, and analog-to-digital converter that is within the scope of the present application.

FIG. 7 illustrates an example of a schematic for a quadruple time-of-flight mass analyzer that uses a single accelerator, two flight tubes, and two reflectrons that is within the scope of the present application.

FIG. 8 shows a computer system that is programmed or otherwise configured to implement methods provided herein.

### DETAILED DESCRIPTION

While various embodiments of the disclosure have been shown and described herein, it will be obvious to those skilled in the art that such embodiments are provided by way of example only. Numerous variations, changes, and substitutions can occur to those skilled in the art without departing from the disclosure. It should be understood that various alternatives to the embodiments of the disclosure described herein can be employed. The section headings used herein are for organizational purposes only and are not to be construed as limiting the subject matter described.

### Definitions

Unless defined otherwise, all technical and scientific terms used herein have the same meaning as is commonly understood by one of skill in the art to which this disclosure belongs. All patents and publications referred to herein are incorporated by reference.

As used herein, “near simultaneous” generally refers to events which occur at nearly, but not exactly the same time. Near simultaneous can refer to parallel events which begin at different times (e.g. with start points within seconds or milliseconds of each other) or can refer to sequential events which occur in rapid succession (e.g. where the end of a first event occurs no more than milliseconds or hundreds of milliseconds before the start of a subsequent event).

As used herein, “quadrupole” generally refers to any device or method step relying on a combination of radiofrequency (or near radiofrequency AC) and DC fields to guide or select ions from an ion flux traversing the AC and/or DC fields. Quadrupoles can take a diverse variety of forms, including but not limited to linear true quadrupoles (i.e. with four straight, parallel, guide rods to which the RF and DC are applied), bent or twisted quadrupoles, hexapoles, octopoles, flatapoles, and the like. Quadrupoles can generally be operated as mass to charge ratio filters or selectors, or as broadband ion guides (e.g. in RF only mode). Ion transmission and/or filtering through a quadrupole can generally be described using a Matthieu stability diagram or calculation.

As used herein, “ion optic” generally refers to any device or combination of devices that are capable of directing the path of an ion flux in a controlled manner. Non limiting examples include AC or DC lenses, quadrupoles, collision cells and the like.

As used herein, “mass analyzer” generally refers to a device which is capable of determining a mass to charge ratio and an intensity or a number of counts per second of one or more ions arriving at a detector comprised within the analyzer.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

The term “biomolecule” refers to biological components that may be involved in corona formation, including, but not limited to, for example, proteins, polypeptides, polysaccharides, a sugar, a lipid, a lipoprotein, a metabolite, an oligonucleotide, metabolome or combination thereof. It is contemplated that the biomolecule coronas of distinct particles may contain some of the same biomolecules, may contain distinct biomolecules with regard to the other sensor elements, and/or may differ in level or quantity, type or conformation of the biomolecule that binds to each sensor element. In one embodiment, the biomolecule is selected from the group of proteins, nucleic acids, lipids, and metabolomes.

As used herein, the terms “amu”, “atomic mass units”, “m/z”, or “mass-to-charge ratio”, when used as a unit of measurement, are used interchangeably and generally refer to a mass-to-charge ratio in Thompsons (Th).

### Multi-Channel Mass Spectrometry Devices

Some embodiments disclosed herein include multi-channel mass spectrometry devices. The multi-channel mass spectrometry devices may have two or more channels for analyzing separate ion fluxes derived from the same sample. The devices may, in some embodiments, provide improved sensitivity, improved analyte detection, faster detection times, improved analyte quantification, and/or other advantages.

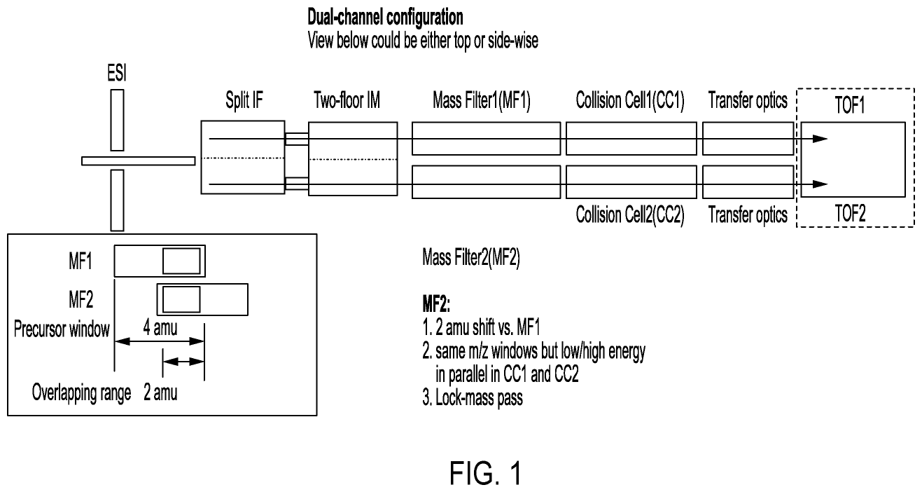

FIG. 1 illustrates an example of a dual-channel configuration for a mass spectrometry device that is within the scope of the present application. From the left, an ion source, such as electrospray ionization (ESI), may deliver ions from a sample to an ion splitter (e.g., split ion funnel) which may split the ion flux into two. The ion splitter may be configured to split the ion flux evenly (e.g., the two ion fluxes exiting the ion splitter are about the same, such as within about 20%, within about 10%, or within about 5%). The separate ion fluxes from the splitter may be received by one or more ion mobility devices that separates the ion flux by mobility. For example, the ion fluxes may each be received by two travelling wave ion mobility devices (TWIMS) that separate the ion fluxes based on mobility. Non-limiting examples of ion mobility devices may be used include drift tubes, high-field asymmetric wave form ion mobility, TIMS, and the like.

The mobility-separated ion fluxes are then received by separate mass filters, such as quadrupoles. The mass filters may be configured to select different ranges of ion masses. As discussed in more detail below, the different ranges of ion masses may, in some embodiments, be selected to have overlapping ranges that may reduce the loss of total ion flux and improve ion detection. As an example, the bottom left box in FIG. 1 illustrates the two mass filters may each select 4 m/z windows that share a 2 m/z overlap. The two mass filters may cycle through several overlapping mass windows. For example, each mass filter may cycle through about 125 windows, each with about a 4 m/z width.

The mass-filtered ion fluxes are received by two collision cells, which may cool and optionally fragment ions before delivering via ion optics to two mass analyzers, such as time-of-flight mass analyzers. The mass analyzers may determine the mass over charge of ions received for the mobility-separated, mass filtered, and optionally fragmented ions.

As a non-limiting example, the dual-channel mass spectrometry device may be coupled to a liquid chromatography device that may separate a sample over time. The time-separated sample can be ionized, and the ion flux separated using the ion splitter. The mass filters may cycle through a desired range of masses for the analytes of interest. For example, the mass filters may select mass windows that cover 400-900 m/z to analyze proteins found in a biological sample. The mass windows may each be 4 m/z wide (125 total windows for each mass filter), and overlap with other mass windows by 2 m/z. The mass filters may repeatedly cycle through the mass windows during the liquid chromatography run time.

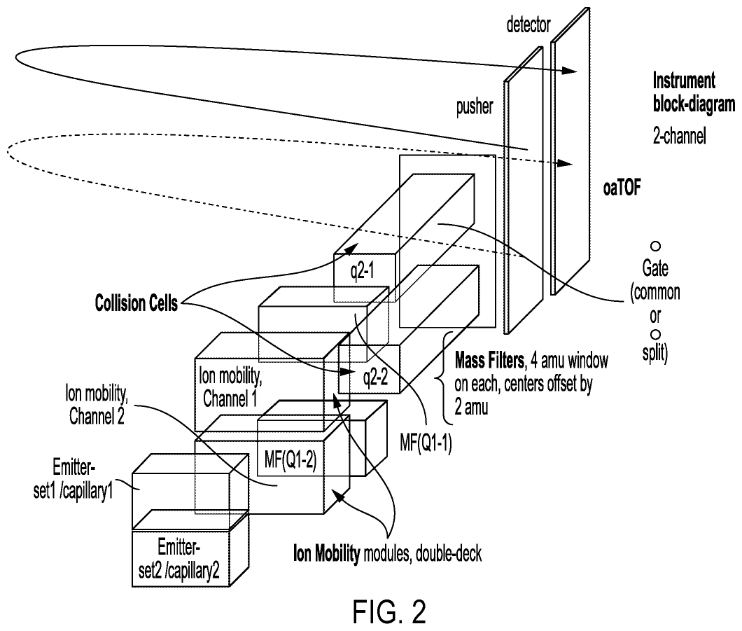

FIG. 2 illustrates an example of a block diagram for a dual-channel configuration for a mass spectrometry device that is within the scope of the present application. In this example, the ion source includes two separate emitters, which can both produce separate ion fluxes from the same sample. For example, both emitters may be coupled to the same liquid chromatography device and receive the same time-separated sample that elutes from the column. Accordingly, in some embodiments, the device may use separate emitters and omit an ion splitter.

The separate ion fluxes from the emitters may be mobility separated using one or more ion mobility devices and then received by the mass filters. The mass filters may select different windows of masses as discussed above. The mass filters, one or both, may optionally operate as RF-only transfer optics elements. The mobility-separated and mass filtered ion fluxes which may cool and optionally fragment the ions before analysis using mass analyzers.

While examples of a multi-channel mass spectrometry devices are provided in FIG. 1 and FIG. 2, numerous variations are possible without departing from the scope of the present application. In some cases, the mass spectrometry devices may have 2, 3, 4, 5, or more channels. This may be implemented, for example, by using an appropriate ion splitter (or multiple ion splitters), or by increasing the number of emitters. Additional components, such as additional mass analyzers and mass filters, can be added to accommodate more channels as needed.

The location of the ion splitter may also vary. In some embodiments, the ion splitter may be between the ion mobility device and the mass filter. For example, a single ion mobility device may be coupled to the ion splitter such that two or more separate ion fluxes of mobility separated ions are delivered to two or more corresponding mass analyzers.

In some embodiments, the ion mobility device(s) may be omitted. For example, the ion splitter may be coupled to two or mass filters without mobility separation.

The mass analyzers may, in some embodiments, detect analytes at about the same time. For example, the mass analyzer may each be time-of-flight mass analyzers that each receive ions from respective collision cells at about the same time and eject the ions into flight tube(s) at about the same time. As discussed further below, the multi-channel mass analyzers may include a time-of-flight mass analyzer where a single accelerator ejects ions received from two collision cells towards separate detectors at about the same time. In some embodiments, the mass analyzers may detect ions at different times. For example, ions from each collision cell are injected into the mass analyzer at different times (e.g., injection into a first mass analyzer and second mass analyzer occur about 50 microseconds to 2 about milliseconds apart).

### Methods of Mass Spectrometry Using Overlapping Windows

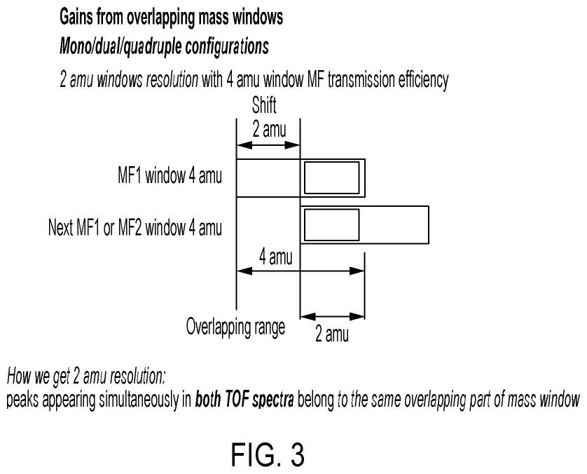

Some embodiments disclosed herein are methods of analyzing a sample using mass spectrometry by analyzing overlapping mass windows. FIG. 3 illustrates an example of using overlapping mass windows to analyze a sample using mass spectrometry that is within the scope of the present application. In this example, overlapping 4 m/z windows are selected and analyzed. For example, one or more quadrupoles may select the two windows, which are each mass analyzed. Peaks associated with analytes that are found in both windows can be associated with the 2 m/z range that is overlapping. Thus, peaks can be assigned to 2 m/z ranges without using 2 m/z windows for the mass filter. Without being bound to any particular theory, this can be advantageous because the ion transmission for the quadrupole may decrease exponentially with small mass windows, and therefore the method may provide narrower mass ranges for analysis without corresponding decreases in ion transmission.

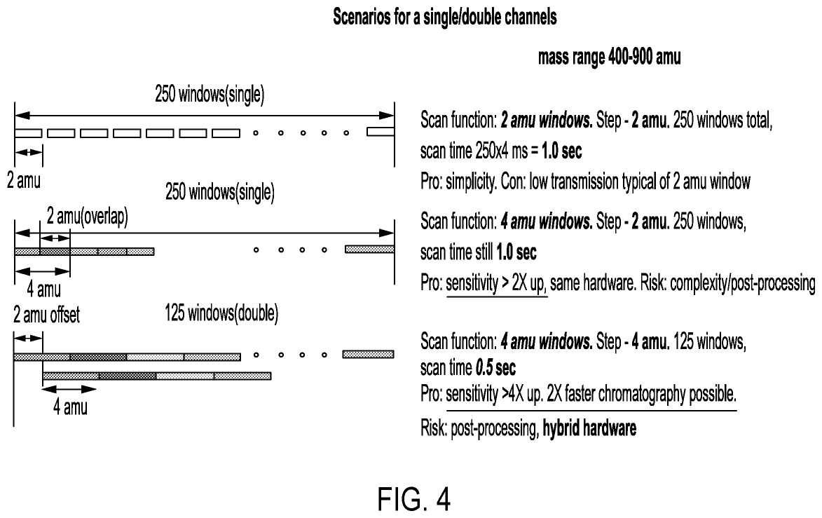

FIG. 4 illustrates an example of different scenarios for analyzing a sample by mass spectrometry by using (i) narrow mass windows in a single channel, (ii) wider, overlapping windows in a single channel, and (iii) wider, overlapping windows in a dual-channel mass spectrometry device. In this example, various analytes are detected in a sample over 400-900 m/z range. The first scenario shows where 2 m/z windows are cycled to detect analytes in a single channel. Assuming each window requires about 4 milliseconds to analyze, the duty cycle will be about 1 second. A disadvantage of the first scenario is that the 2 m/z windows can result in low ion transmission, which can reduce sensitivity. The second scenario shows where 4 m/z windows are cycled, but with a step of 2 m/z such that there is a 2 m/z overlap between each window. The duty cycle remains 1 second. This allows for improved ion transmission, and the larger windows may still be used to associated analytes to 2 m/z windows as discussed above. The third scenario shows using the same overlapping windows in a dual-channel device, but in this case overlapping occurs between mass windows in mass filters on separate channels. As no overlapping happens in individual channels, the reduced number of windows in each channel results in reduced duty cycle. In this case, there is improved ion transmission and association of analytes with 2 m/z windows, but the duty cycle may be reduced to 0.5 seconds. This may allow for reduced run times for liquid chromatography or additional datapoints for each chromatography peak. Otherwise, if the duty cycle remains 1.0 sec, the time for ion collection in each mass window may be increased two-fold, e.g. from 4 ms to 8 ms, resulting in better sensitivity.

While examples are discussed for overlapping windows, numerous variations are possible without departing from the scope of the present application. The size of the overlapping windows may be selected based upon the desired application. For example, the windows may be about 3 m/z to about 10 m/z (e.g., about 3 m/z, about 4 m/z, about 5 m/z, about 6 m/z, about 7 m/z, about 8 m/z, about 9 m/z, or about 10 m/z). Also, the degree of overlap between windows may be about half the width, but other amounts of overlap are possible. For example, each window may overlap by about one third, and the windows are stepped by one third of the window each time (e.g., 3 m/z windows are stepped by 1 m/z). In this case, peaks common to three overlapping windows may be associated with a 1 m/z range. Numerous other variants are possible. In some embodiments, the step between each window analyzed is about the same as the mass range that is overlapping between the windows.

In some embodiments, the methods disclosed herein may be implemented using a single-channel mass spectrometer (e.g., using commercially available mass spectrometers, such as the ORBITRAP EXPLORIS 480). In some embodiments, the methods disclosed herein may be implemented using a multi-channel mass spectrometer such as those disclosed in the present application (e.g., the dual-channel mass spectrometers depicted in FIG. 1 and FIG. 2). In cases, the multi-channel mass spectrometer may provide increased sensitivity and/or reduced duty cycle when implementing the method.

In some embodiments, the overlapping windows may be varied over time. In some embodiments, the overlapping windows may varied based on the anticipated mass range of analytes at a given elution time. For example, analytes around a first elution time may be anticipated to have masses from 400-600 m/z, while analytes around a second elution time may be anticipated to have masses from 500-700 m/z. Different overlapping mass windows may be used at the different elution time that correspond to these anticipated masses. In some embodiments, the mass ranges are adjusted based on the anticipated or measured ion mobility of ions. For example, different mass ranges may be selected based on the mobility of ions received from the ion mobility device. In some embodiments, the window size may be varied based on an expected amount of analytes (e.g., during various elution times and/or masses). For example, when a greater number of analytes are expected, the windows may be smaller (e.g., overlapping 4 m/z windows), and when less analytes are expected the windows may be larger (e.g., overlapping 8 m/z windows).

When the method is implemented using a multi-channel mass spectrometry device, the windows may be distributed among the two or more mass analyzers in various ways. For example, two mass filters may each select 4 m/z windows spanning 400-900 m/z, where each mass filters steps 4 m/z, but the windows for each mass filter are offset by 2 m/z. Thus, mass data from one window from the first mass filter and mass data from one window of the second mass filter are used to identify peaks in each of the overlapping 2 m/z range. As an alternative example, a first mass filter may select 4 m/z windows with 2 m/z steps spanning 400-650 m/z, and a second mass filter may select 4 m/z windows with 2 m/z steps spanning 650-900 m/z. Thus, mass data for two windows from the same mass filter are used to identify peaks in each of the overlapping 2 m/z range.

In some embodiments, two mass window that overlap are analyzed using the mass analyzer within less than 1 second, less than 500 milliseconds, less than 50 milliseconds, less than 25 milliseconds, less than 15 milliseconds, less than 10 milliseconds, or less than 5 milliseconds. In some embodiments, two mass window that overlap are analyzed using the mass analyzer at about the same time. For example, a mass window of 500-504 m/z is analyzed in the first mass analyzer and a mass window of 502-506 is analyzed in the second mass analyzer at about the same time.

In some embodiments, the method may further comprise identifying the analyte using common peaks found in overlapping mass ranges. In some embodiments, the analytes are fragmented in a collision cell before mass analysis. In some embodiments, common peaks for overlapping mass windows can be associated with the overlapping mass range for the precursor ion. In some embodiments, the precursor ion mass range and the fragmented ion masses are used to identify the analyte. As a non-limiting example, the analyte may be a peptide, and the mass of the peptide may be associated with a 2 m/z mass range using data from the overlapping 4 m/z mass windows. This narrower window further limits the search space of possible peptides that can be associated with the fragment pattern, which may in turn improve detection and/or quantification. In some cases, the narrow mass range may only contain peaks associated with a single analyte, whereas the larger mass windows may have peaks associated with two or more analytes that may reduce detection and/or quantification.

Some embodiments disclosed herein include a computer readable medium comprising machine-executable code that, upon execution by the one or more computer processors, implements one or more the methods disclosed herein. In some cases, the method comprises obtaining data representing signal intensity against m/z for a plurality of overlapping mass windows, comparing the overlapping windows to associate peaks with a narrower mass range that overlaps between two or more mass windows, and identifying one or more analytes based on the narrower mass range and the peaks associated with the narrower mass range.

In some embodiments, identifying one or more analytes includes comparing a library of analyte mass data with the narrower mass range and the peaks associated with the narrower mass range. In some embodiments, a probability of detection for an analyte within the library of analyte mass data based on the narrower mass range and the peaks associated with the narrower mass range. In some embodiments, an analyte is identified if the probability of detection exceeds a threshold (e.g., a pre-determined threshold).

### Parallel Mass Analyzers

Some embodiment disclosed herein include a parallel mass analyzer. The parallel mass analyzer may allow for separate detection of analytes in two or more different ion populations using shared components, such as a common flight tube and reflectron. In some embodiments, the parallel mass analyzer may be used in the multi-channel mass spectrometer devices disclosed herein.

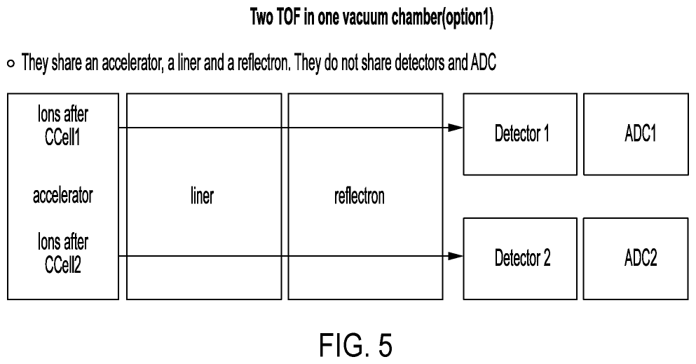

FIG. 5 illustrates an example of a schematic for a dual time-of-flight mass analyzer that shares a common flight tube, accelerator, and reflectron that is within the scope of the present application. As depicted, an accelerator receives two populations of ions from two collision cells. For example, the two collision cells may be those from the dual-channel mass spectrometry device as discussed above (e.g., the devices depicted in FIG. 1 or FIG. 2), where separate ion channels are mass filtered before collision cells transfer the separate channels to mass analyzers. The two populations of ion are spaced apart within the accelerator. The accelerator may eject the two populations of ions into the flight tube (liner), and the ion mirror (reflection) may direct the populations of ions to separate detectors. Each detector can be operably coupled to separate signal converters (e.g., analog-to-digital converter). Thus, the different population of ions can be separately detected while still using a single accelerator, flight tube, and reflectron, which may advantageously reduce the cost, reduce footprint, and/or reduce vacuum requirements.

In some embodiments, each ion population comprises a different mass window of ions derived from the same sample. For example, as discussed above, the first population of ions may have a first mass window of ions, and the second population of ions may have a second mass window of ions that overlaps with the first mass window. The signal received from the two detectors may be used to identify analytes associated with the narrower mass range overlapping between the two mass windows. As another example, the first population of ions may have a first mass window of non-fragmented ions (precursors), and the second population of ions are fragment ions from the same window of precursor ions. The signal received from the two detectors may be used to identify analytes associated with the narrower mass range overlapping between the two mass windows. In some embodiments, each ion population comprises a different mass window of ions derived from the same sample, where both mass windows comprise fragmented ions.

In some embodiments, the accelerator ejects both populations of ions into the flight tube at about the same time. In some embodiments, the accelerator ejects each population of ions into the flight tube at different times (e.g., sequentially). As an example, a first collision cell injects a first populations of ions into the accelerator, and after the first population of ions are ejected into the flight tube, the second collision cell injects a second population of ions into the accelerator which are then ejected into the flight tube after the first population reached the detector. This cycle can be repeated.

In some embodiments, the parallel mass analyzer may comprise a single vacuum chamber.

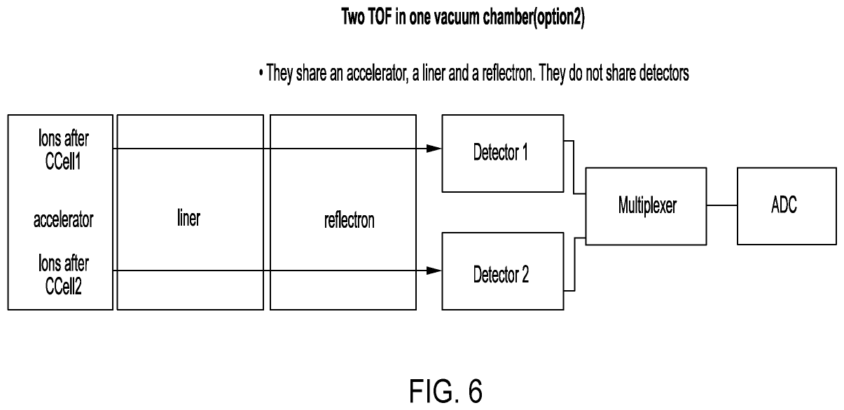

FIG. 6 illustrates an example of a schematic for a dual time-of-flight mass analyzer that shares a common flight tube, accelerator, reflectron, and analog-to-digital converter that is within the scope of the present application. As depicted, the dual-channel time-of-flight mass analyzer shares the same components and configuration as discussed above, except that the detectors share a common signal converter operably coupled via a multiplexer. In this case, ions from the two collision cells may be ejected into the flight tube at different times. The signal converter alternates between receiving signals from the two detectors depending on which detector is receiving ions at the time.

In some embodiments, the two detectors may share common components. As discussed above, they may share a common signal converter. In some embodiments, the detector may share a common microchannel plate. The microchannel plate may have a first region configured to receive a first population of ions from the first collision cell, and a second region configured to receive a second population of ions from the second collision cell. In some embodiments, a first anode is configured to receive ions from the first region of the plate, and a second anode is configured to receive ions from the second region of the plate. The signals from the two anodes can be used to separately identify analytes from the first population and the second population.

While parallel mass analyzer have been described with two channels for analyzing ions, the mass analyzer can be configured with additional components to increase the number of channels. In some embodiments, the parallel mass analyzer may have 2, 3, 4, or more channels. For example, 4 channels may include four separate collision cells that inject ions into the accelerator at different regions, where the ion populations are spaced apart within the accelerator. Four detectors may receive ions from the respective ion populations.

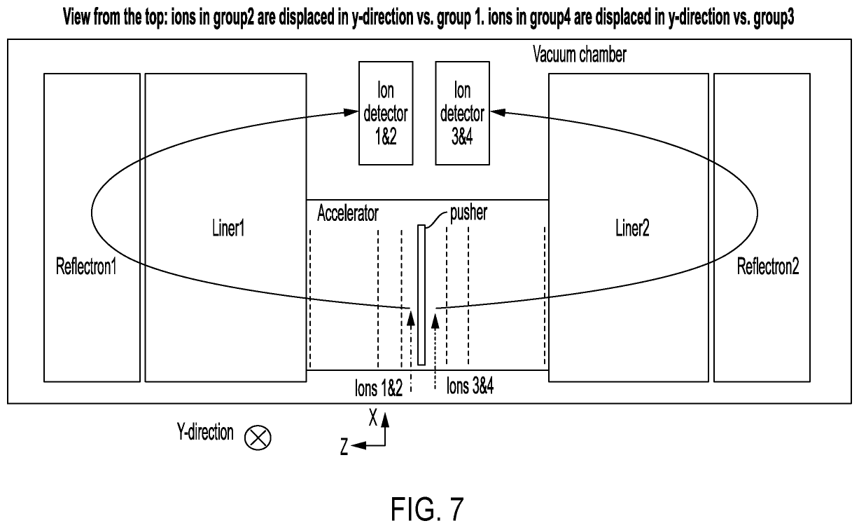

FIG. 7 illustrates an example of a schematic for a quadruple time-of-flight mass analyzer that uses a single accelerator, two flight tubes, and two reflectrons that is within the scope of the present application. The schematic shows the path of two ion populations along a plane that is perpendicular to the y-direction. Paths for the second pair of ion populations are offset in the y-direction (not shown). As depicted, four populations are injected into the accelerator and are spaced apart. Two of the populations of ions are injected on each side of the pusher. A potential is applied to the pusher to eject the ions into the flight tubes, where the two ion populations on the same side of the pusher (ions 1&2) are ejected into the same flight tube. The accelerator may also comprise grids (dashed lines) to provide a more uniform electric field. Each of the ion populations are directed by ion mirror to a respective detector.

In some embodiments, the parallel mass analyzer may be contained in a single vacuum chamber.

The quadruple time-of-flight mass analyzer may include various additional features or modifications without departing from the scope of the present application. For example, the configuration may be modified to accommodate 6 or 8 channels (3 or 4 pairs of ion populations on each side of the pusher). As another example, all the populations of ions may be ejected into the flight tubes at the same time or different times. As another example, the detectors may share common components, such as microchannel plates and/or signal converters.

The parallel mass analyzers disclosed herein may, in some embodiments, be used in the multi-channel mass spectrometer devices disclosed herein. For example, the two mass analyzers depicted in FIG. 1 and FIG. 2, may be a dual time-of-flight mass analyzer as depicted in FIG. 5. The parallel mass analyzer may also be used in other configurations where more than one mass analyzer can be used.

### Computer Systems

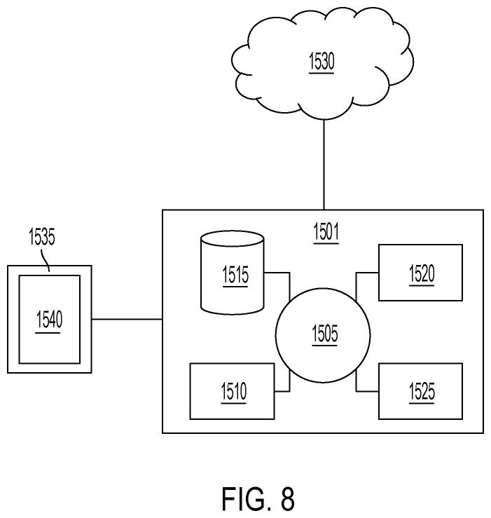

The present disclosure provides computer systems that are programmed to implement methods of the disclosure. FIG. 8 shows a computer system 1501 that is programmed or otherwise configured to implement the methods and systems described herein. The computer system 1501 can be an electronic device of a user or a computer system that is remotely located with respect to the electronic device. The electronic device can be a mobile electronic device.

The computer system 1501 includes a central processing unit (CPU, also “processor” and “computer processor” herein) 1505, which can be a single core or multi core processor, or a plurality of processors for parallel processing. The computer system 1501 also includes memory or memory location 1510 (e.g., random-access memory, read-only memory, flash memory), electronic storage unit 1515 (e.g., hard disk), communication interface 1520 (e.g., network adapter) for communicating with one or more other systems, and peripheral devices 1525, such as cache, other memory, data storage and/or electronic display adapters. The memory 1510, storage unit 1515, interface 1520 and peripheral devices 1525 are in communication with the CPU 1505 through a communication bus (solid lines), such as a motherboard. The storage unit 1515 can be a data storage unit (or data repository) for storing data. The computer system 1501 can be operatively coupled to a computer network (“network”) 1530 with the aid of the communication interface 1520. The network 1530 can be the Internet, an internet and/or extranet, or an intranet and/or extranet that is in communication with the Internet. The network 1530 in some cases is a telecommunication and/or data network. The network 1530 can include one or more computer servers, which can enable distributed computing, such as cloud computing. The network 1530, in some cases with the aid of the computer system 1501, can implement a peer-to-peer network, which may enable devices coupled to the computer system 1501 to behave as a client or a server.

The CPU 1505 can execute a sequence of machine-readable instructions, which can be embodied in a program or software. The instructions may be stored in a memory location, such as the memory 1510. The instructions can be directed to the CPU 1505, which can subsequently program or otherwise configure the CPU 1505 to implement methods of the present disclosure. Examples of operations performed by the CPU 1505 can include fetch, decode, execute, and writeback.

The CPU 1505 can be part of a circuit, such as an integrated circuit. One or more other components of the system 1501 can be included in the circuit. In some cases, the circuit is an application specific integrated circuit (ASIC).

The storage unit 1515 can store files, such as drivers, libraries and saved programs. The storage unit 1515 can store user data, e.g., user preferences and user programs. The computer system 1501 in some cases can include one or more additional data storage units that are external to the computer system 1501, such as located on a remote server that is in communication with the computer system 1501 through an intranet or the Internet.

The computer system 1501 can communicate with one or more remote computer systems through the network 1530. For instance, the computer system 1501 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 1501 via the network 1530.

Methods as described herein can be implemented by way of machine (e.g., computer processor) executable code stored on an electronic storage location of the computer system 1501, such as, for example, on the memory 1510 or electronic storage unit 1515. The machine executable or machine-readable code can be provided in the form of software. During use, the code can be executed by the processor 1505. In some cases, the code can be retrieved from the storage unit 1515 and stored on the memory 1510 for ready access by the processor 1505. In some situations, the electronic storage unit 1515 can be precluded, and machine-executable instructions are stored on memory 1510.

The code can be pre-compiled and configured for use with a machine having a processor adapted to execute the code or can be compiled during runtime. The code can be supplied in a programming language that can be selected to enable the code to execute in a pre-compiled or as-compiled fashion.

Aspects of the systems and methods provided herein, such as the computer system 1501, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1501 can include or be in communication with an electronic display 1535 that comprises a user interface (UI) 1540 for providing, for example, selection of ion manifold injection parameters. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 1505.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the present disclosure may be employed in practicing the present disclosure. It is intended that the following claims define the scope of the present disclosure and that methods and structures within the scope of these claims and their equivalents be covered thereby. The section headings used herein are for organizational purposes only and are not to be construed as limiting the subject matter described.

### EMBODIMENTS

The following are exemplary embodiments of the disclosure herein:

Embodiment 1. An apparatus for parallel mass spectrometry analysis comprising:
one or more ion mobility devices configured to receive ions from an ion source;
a first mass filter configured to receive mobility-separated ions from the one or more ion mobility devices;
a second mass filter configured to receive mobility-separated ions from the one or more ion mobility devices;
a first collision cell configured to receive mass-filtered ions from the first mass filter;
a second collision cell configured to receive mass-filtered ions from the second mass filter;
a first mass analyzer configured to analyze ions received from the first collision cell; and
a second mass analyzer configured to analyze ions received from the second collision cell.

Embodiment 2. The apparatus of embodiment 1, wherein the first collision cell is configured to fragment the mass-filtered ions from the first mass filter, and wherein the first mass analyzer is configured to analyze the fragmented ions received from the first mass filter.

Embodiment 3. The apparatus of any one of embodiments 1-2, wherein the second collision cell is configured to fragment the mass-filtered ions from the second mass filter, and wherein the second mass analyzer is configured to analyze the fragmented ions received from the second mass filter.

Embodiment 4. The apparatus of any one of embodiments 1-3, wherein the ion source comprises an electrospray ionization device.

Embodiment 5. The apparatus of any one of embodiments 1-2, wherein the one or more ion mobility devices comprise a first ion mobility device and a second ion mobility device, wherein the first mass filter is configured to receive mobility-separated ions from the first ion mobility device, and wherein the second mass filter is configured to receive mobility-separate ions from the second ion mobility device.

Embodiment 6. The apparatus of embodiment 5, wherein the apparatus further comprises a split ion funnel, wherein the split ion funnel is configured to direct a first portion of the ions from the ion source to the first ion mobility device and a second portion of the ions from the ion source to the second ion mobility device.

Embodiment 7. The apparatus of any one of embodiment 5-6, where the ion source comprises a first electrospray ionization device and second electrospray ionization, wherein the first ion mobility device is configured to receive ions from the first electrospray ionization device, and wherein the second ion mobility is configured to receive ion from the second electrospray ionization device.

Embodiment 8. The apparatus of any one of embodiments 5-7, wherein both the first ion mobility device and the second ion mobility device are disposed in a first common vacuum chamber.

Embodiment 9. The apparatus of any one of embodiments 1-8, wherein both the first mass analyzer and the second mass analyzer are disposed in a second common vacuum chamber.

Embodiment 10. The apparatus of any one of embodiments 1-9, wherein the ion mobility devices comprise a travelling wave ion mobility device.

Embodiment 11. The apparatus of any one of embodiments 5-10, wherein the first ion mobility device comprises a first travelling wave ion mobility devices, and wherein the second ion mobility device comprises a second travelling wave ion mobility device.

Embodiment 12. The apparatus of embodiment 11, wherein the first travelling wave ion mobility device and the second travelling wave ion mobility device share at least one common substrate comprising a plurality of electrodes.

Embodiment 13. The apparatus of any one of embodiments 1-12, wherein the mobility-separated ions are separated using a mobility path of at least 10 m.

Embodiment 14. The apparatus of any one of embodiments 1-13, wherein the apparatus further comprises:
a third mass filter configured to receive mobility-separated ions from the one or more ion mobility devices;
a third collision cell configured to receive mass-filtered ions from the third mass filter; and
a third mass analyzer configured to analyze ions received from the third collision cell.

Embodiment 15. The apparatus of any one of embodiments 1-13, wherein the apparatus further comprises: a third mass filter configured to receive mobility-separated ions from the one or more ion mobility devices; and a third mass analyzer configured to analyze unfragmented ions received from the third mass filter.

Embodiment 16. The apparatus of any one of embodiments 14-15, wherein the apparatus further comprises:
a fourth mass filter configured to receive mobility-separated ions from the one or more ion mobility devices;
a fourth collision cell configured to receive mass-filtered ions from the fourth mass filter; and
a fourth mass analyzer configured to analyze ions received from the fourth collision cell.

Embodiment 17. The apparatus of any one of embodiments 1-15 further comprising a liquid chromatography device in fluid communication with the ion source.

Embodiment 18. The apparatus of any one of embodiments 1-17, wherein the first mass analyzer comprises a time-of-flight mass analyzer.

Embodiment 19. The apparatus of any one of embodiments 1-18, wherein the second mass analyzer comprises a time-of-flight mass analyzer.

Embodiment 20. The apparatus of any one of embodiments 1-19, wherein the first mass filter is configured to select first mass windows, wherein the second mass filter is configured to select second mass windows, wherein the first mass windows and the second mass windows are different, and wherein the first mass windows and the second mass windows overlap.

Embodiment 21. The apparatus of embodiment 20, wherein the first mass windows are each less than 20 m/z, less than 15 m/z, less than 10 m/z, or less than 5 m/z.

Embodiment 22. The apparatus of any one of embodiments 20-21, wherein the first mass windows are each greater than 1.5 m/z, greater than 2 m/z, greater than 3 m/z or greater than 4 m/z.

Embodiment 23. The apparatus of any one of embodiments 20-22, wherein the first mass windows are non-overlapping.

Embodiment 24. The apparatus of any one of embodiments 20-23, wherein the first mass windows overlap by no more than 20%, no more than 15%, no more than 10%, or no more than 5%.

Embodiment 25. The apparatus of any one of embodiments 20-24, wherein the first mass windows are each about the same size.

Embodiment 26. The apparatus of any one of embodiments 20-25, wherein the second mass windows are each less than 20 m/z, less than 15 m/z, less than 10 m/z, or less than 5 m/z.

Embodiment 27. The apparatus of any one of embodiments 20-26, wherein the second mass windows are each greater than 1.5 m/z, greater than 2 m/z, greater than 3 m/z or greater than 4 m/z.

Embodiment 28. The apparatus of any one of embodiments 20-27, wherein the second mass windows are non-overlapping.

Embodiment 29. The apparatus of any one of embodiments 20-28, wherein the first mass windows overlap by no more than 20%, no more than 15%, no more than 10%, or no more than 5%.

Embodiment 30. The apparatus of any one of embodiments 20-29, wherein the first mass windows together form a continuous range of at least 50 m/z, at least 100 m/z, at least 150 m/z, at least 200 m/z, at least 400 m/z or at least 500 m/z.

Embodiment 31. The apparatus of any one of embodiments 20-30, wherein the second mass windows are each about the same size.

Embodiment 32. The apparatus of any one of embodiments 20-31, wherein the first mass windows and the second mass windows overlap.

Embodiment 33. The apparatus of embodiment 32, wherein the first mass windows overlap with the second mass windows by at least 30%, at least 40%, at least 50%, at least 75, or at least 90%.

Embodiment 34. The apparatus of embodiments 32 or 33, wherein the first mass windows and the second mass windows together form a continuous window of at least about 200 m/z, at least about 250 m/z, at least about 300 m/z, at least about 350 m/z, at least about 400 m/z, at least about 450 m/z or at least 500 m/z.

Embodiment 35. The apparatus of any one of embodiments 20-34, the first mass windows comprise at least 100 windows.

Embodiment 36. The apparatus of any one of embodiments 20-35, the second mass windows comprise at least 100 windows.

Embodiment 37. The apparatus of any one of embodiments 20-36, wherein the first mass filter is configured to cycle through the first mass windows in less than about 2 seconds.

Embodiment 38. The apparatus of any one of embodiments 20-37, wherein the second mass filter is configured to cycle through the second mass windows in less than about 2 seconds.

Embodiment 39. The apparatus of any one of embodiments 20-38, wherein the first mass filter is configured to cycle through the first mass windows at about the same time as the second mass filter is configured to cycle through second mass windows.

Embodiment 40. The apparatus of any one of embodiments 20-39, wherein the first mass windows and the second mass windows change based on the mobility of ions received from the one or more ion mobility devices.

Embodiment 41. The apparatus of any one of embodiments 20-40, wherein each of the first mass windows are different from each of the second mass windows.

Embodiment 42. The apparatus of any one of embodiments 1-41, wherein the first collision cell is configured to fragment ions using different conditions than the second collision cell.

Embodiment 43. The apparatus of any one of embodiments 1-41, wherein the first collision cell is configured to fragment ions using about the same conditions as the second collision cell.

Embodiment 44. The apparatus of any one of embodiments 1-43, wherein the ion source is operably coupled to a liquid chromatography device, and wherein both the first mass analyzer and the second mass analyzer are configured to receive ions derived from a sample separated by the liquid chromatography device.

Embodiment 45. A method for parallel mass spectrometry analysis comprising:
introducing gas-phase ions obtained from a sample into one or more ion mobility devices;
sequentially introducing windows of ion mobilities from the one or more ion mobility devices to a first mass filter;
sequentially introducing windows of ion mobilities from the one or more ion mobility devices to a second mass filter;
sequentially introducing windows of ion masses from the first mass filter to a first collision cell;
sequentially introducing windows of ion masses from the second mass filter to a second collision cell;
sequentially analyzing windows of ions received from the first collision cell using a first mass analyzer; and
sequentially analyzing windows of ions received from the second collision cell using a second mass analyzer.

Embodiment 46. The method of embodiment 45, wherein the gas-phase ions are introduced using an electrospray ionization device.

Embodiment 47. The method of any one of embodiments 45-46, wherein the one or more ion mobility devices comprise a first ion mobility device and a second ion mobility device, and wherein the method comprises:
introducing a first portion of the gas-phase ions into the first ion mobility device;
introducing a second portion of the gas-phase ions into the second ion mobility device;
sequentially introducing windows of ion mobilities from the first ion mobility device to the first mass filter; and
sequentially introducing windows of ion mobilities from the second ion mobility device to the second mass filter.

Embodiment 48. The method of embodiment 47, wherein the method comprises using a split ion funnel to direct the first portion of the ions from the ion source to the first ion mobility device and the second portion of the ions from the ion source to the second ion mobility device.

Embodiment 49. The method of any one of embodiments 47-48, where the ion source comprises a first electrospray ionization device and second electrospray ionization, wherein the method comprises:
introducing gas-phase ions from the first electrospray ionization device to the first ion mobility device; and
introducing gas-phase ions from the second electrospray ionization device to the second ion mobility device.

Embodiment 50. The method of any one of embodiments 47-49, wherein both the first ion mobility device and the second ion mobility device are disposed in a first common vacuum chamber.

Embodiment 51. The method of any one of embodiments 45-50, wherein both the first mass analyzer and the second mass analyzer are disposed in a second common vacuum chamber.

Embodiment 52. The method of any one of embodiments 45-51, wherein the windows of ion mobilities are obtained using travelling wave ion mobility.

Embodiment 53. The method of any one of embodiments 47-52, wherein the first ion mobility device comprises a first travelling wave ion mobility device, and wherein the second ion mobility device comprises a second travelling wave ion mobility device.

Embodiment 54. The method of embodiment 53, wherein the first travelling wave ion mobility device and the second travelling wave ion mobility device share at least one common substrate comprising a plurality of electrodes.

Embodiment 55. The method of any one of embodiments 45-54, wherein the method comprising obtaining windows of mobility-separated ions are separated using a mobility path of at least 10 m.

Embodiment 56. The method of any one of embodiments 45-55, wherein the method comprises:
sequentially introducing windows of ion mobilities from the one or more ion mobility devices to a third mass filter;
sequentially introducing windows of ion masses from the third mass filter to a third collision cell; and
sequentially analyzing windows of ions received from the third collision cell using a third mass analyzer.

Embodiment 57. The method of any one of embodiments 45-56, wherein the method comprises:
sequentially introducing windows of ion mobilities from the one or more ion mobility devices to a third mass filter; and
sequentially analyzing windows of unfragmented ions received from the mass filter using a third mass analyzer.

Embodiment 58. The method of any one of embodiments 56-57, wherein the method comprises:
sequentially introducing windows of ion mobilities from the one or more ion mobility devices to a fourth mass filter;
sequentially introducing windows of ion masses from the fourth mass filter to a fourth collision cell; and
sequentially analyzing windows of ions received from the fourth collision cell using a fourth mass analyzer.

Embodiment 59. The method of any one of embodiments 45-57, wherein the method comprises separating compounds from a sample over time and introducing the time-separated compounds to the ion source.

Embodiment 60. The method of embodiment 58, wherein the compounds are separated by liquid chromatography.

Embodiment 61. The method of any one of embodiments 45-60, wherein the first mass analyzer comprises a time-of-flight mass analyzer.

Embodiment 62. The method of any one of embodiments 45-61, wherein the second mass analyzer comprises a time-of-flight mass analyzer.

Embodiment 63. The method of any one of embodiments 45-62, wherein the first mass filter selects first mass windows, wherein the second mass filter selects second mass windows, wherein the first mass windows and second mass windows are different, and wherein the first mass windows and the second mass windows overlap.

Embodiment 64. The method of embodiment 63, wherein the first mass windows are each less than 20 m/z, less than 15 m/z, less than 10 m/z, or less than 5 m/z.

Embodiment 65. The method of any one of embodiments 63-64, wherein the first mass windows are each greater than 1.5 m/z, greater than 2 m/z, greater than 3 m/z or greater than 4 m/z.

Embodiment 66. The method of any one of embodiments 63-65, wherein the first mass windows are non-overlapping.

Embodiment 67. The method of any one of embodiments 63-65, wherein the first mass windows overlap by no more than 20%, no more than 15%, no more than 10%, or no more than 5%.

Embodiment 68. The method of any one of embodiments 63-67, wherein the first mass windows are each about the same size.

Embodiment 69. The method of any one of embodiments 63-68, wherein the second mass windows are each less than 20 m/z, less than 15 m/z, less than 10 m/z, or less than 5 m/z.

Embodiment 70. The method of any one of embodiments 63-69, wherein the second mass windows are each greater than 1.5 m/z, greater than 2 m/z, greater than 3 m/z or greater than 4 m/z.

Embodiment 71. The method of any one of embodiments 63-70, wherein the second mass windows are non-overlapping.

Embodiment 72. The method of any one of embodiments 63-71, wherein the second mass windows overlap by no more than 20%, no more than 15%, no more than 10%, or no more than 5%.

Embodiment 73. The method of any one of embodiments 63-72, wherein the second mass windows are each about the same size.

Embodiment 74. The method of any one of embodiments 63-73, wherein the first mass windows and the second mass windows overlap.

Embodiment 75. The method of embodiment 74, wherein the first mass windows overlap with the second mass windows by at least 30%, at least 40%, at least 50%, at least 75, or at least 90%.

Embodiment 76. The method of embodiments 74 or 75, wherein the first mass windows and the second mass windows together form a continuous window of at least about 200 m/z, at least about 250 m/z, at least about 300 m/z, at least about 350 m/z, at least about 400 m/z, at least about 450 m/z or at least about 500 m/z.

Embodiment 77. The method of any one of embodiments 63-76, the first mass windows comprise at least 100 windows.

Embodiment 78. The method of any one of embodiments 63-77, the second mass windows comprise at least 100 windows.

Embodiment 79. The method of any one of embodiments 63-78, wherein the first mass filter cycles through the first mass windows in less than about 2 seconds.

Embodiment 80. The method of any one of embodiments 63-79, wherein the second mass filter cycles through the second mass windows in less than about 2 seconds.

Embodiment 81. The method of any one of embodiments 63-80, wherein the first mass windows and the second mass windows change based on the mobility of ions received from the one or more ion mobility devices.

Embodiment 82. The method of any one of embodiments 63-81, wherein pairs of mass windows that overlap are both analyzed within less than 1 second, less than 500 milliseconds, less than 50 milliseconds, less than 25 milliseconds, less than 15 milliseconds, less than 10 milliseconds, or less than 5 milliseconds.

Embodiment 83. The method of any one of embodiments 45-82, wherein the first collision cell fragments ions using different conditions than the second collision cell.

Embodiment 84. The method of any one of embodiments 45-82, wherein the first collision cell fragments ions using about the same conditions as the second collision cell.

Embodiment 85. The method of any one of embodiments 45-82, wherein the sample is derived from a biofluid.

Embodiment 86. A parallel mass analyzer comprising:
a flight tube;
an ion mirror;
one or more ion detectors;
one or more ion optics elements;
an accelerator configured to eject first ions and second ions into the flight tube, wherein the first ions are directed by the ion mirror to a first region of the one or more detectors, and wherein the second ions are directed by the ion mirror to a second region of the one or more ion detectors.

Embodiment 87. The parallel mass analyzer of embodiment 86, wherein the first ions and the seconds ions are separately detected by the one or more detectors.

Embodiment 88. The parallel mass analyzer of any one of 86-87, wherein the accelerator is configured to receive the first ions from a first collision cell and the second ions from a second collision cell.

Embodiment 89. The parallel mass analyzer of any one of embodiments 86-88, wherein the accelerator is configured to receive the first ions and the second ions at about the same time.

Embodiment 90. The parallel mass analyzer of any one of embodiments 86-88, wherein the accelerator is configured to receive the first ions and the second ions at different times.

Embodiment 91. The parallel mass analyzer of any one of embodiments 86-89, wherein the accelerator is configured to eject the first ions and the second ions into the flight tube at about the same time.

Embodiment 92. The parallel mass analyzer of any one of embodiments 86-90, wherein the accelerator is configured to eject the first ions and the second ions into the flight tube at different times.

Embodiment 93. The parallel mass analyzer of embodiment 92, wherein the one or more detectors are operably coupled to a common signal converter, wherein the common signal converter converts signals associated with ions received at the first region and signals associated with ions received at the second region.

Embodiment 94. The parallel mass analyzer of embodiment 93, wherein the common signal converter is an analog-to-digital converter.

Embodiment 95. The parallel mass analyzer of embodiments 93 or 91, wherein the signal converter is operably coupled to the one or more detectors via a multiplexer.

Embodiment 96. The parallel mass analyzer of any one of embodiments 86-95, wherein the accelerator is configured to eject the first ions into the flight tube along a first path, wherein the accelerator is configured to eject the second ions into the flight tube along a second path, and wherein the first path and the second path are spaced apart.

Embodiment 97. The parallel mass analyzer of embodiment 96, wherein the spacing between the first path and the second path is about 4 mm to 20 mm, 5 mm to 15 mm and 8 mm to 12 mm.

Embodiment 98. The parallel mass analyzer of embodiments 96 or 97, wherein the first path and the second path are generally parallel.

Embodiment 99. The parallel mass analyzer of any one of 96-98, wherein the first region and the second region are spaced apart by at least about 5 mm, at least about 8 mm, at least about 10 mm, or at least about 20 mm.

Embodiment 100. The parallel mass analyzer of any one of embodiments 85-99, wherein the accelerator is configured to receive the first ions from a first collision cell and the second ions from a second collision cell.

Embodiment 101. The parallel mass analyzer of any one of embodiments 86-100, wherein the one or more ion detectors comprise one ion detector.

Embodiment 102. The parallel mass analyzer of embodiment 101, wherein the ion detector comprises:
a microchannel plate;
a first anode operably coupled to the microchannel plate and configured to receive the first ions directed to the first region of the ion detector; and
a second anode operably coupled to the microchannel plate and configured to receive the second ions directed to the second region of the ion detector.

Embodiment 103. The parallel mass analyzer of any one of embodiments 86-100, wherein the one or more ion detectors comprise:
a first ion detector configured to receive the first ions directed to the first region; and
a second ion detector configured to receive the second ions directed to the second region.

Embodiment 104. The parallel mass analyzer of any one of embodiments 86-103, wherein the first ions and the second ions are derived from the same sample.

Embodiment 105. The parallel mass analyzer of any one of embodiments 86-104, wherein the first ions and the second ions have different ion mobilities.

Embodiment 106. The parallel mass analyzer of any one of embodiments 86-105, wherein the first region of the one or more ion detectors and the second region of the one or more ion detectors are spaced apart.

Embodiment 107. The parallel mass analyzer of any one of embodiments 86-105, wherein the first region of the one or more ion detectors and the second region of the one or more ion detectors are spaced apart by at least 5 mm, at least 10 mm, at least 20 mm, or at least 50 mm.

Embodiment 108. The parallel mass analyzer of any one of embodiments 86-107, wherein the ion mirror is a single-stage ion mirror, a dual-stage ion mirror, or a multi-stage ion mirror.

Embodiment 109. The parallel mass analyzer of any one of embodiments 86-108, wherein the ion mirror is gridless.

Embodiment 110. The parallel mass analyzer of any one of embodiments 86-109, further comprising a second flight tube, a second ion mirror and a second one or more detectors, wherein the accelerator is configured to eject third ions and fourths ions into the second flight tube, wherein the third ions are directed by the second ion mirror to a first region of the second one or more detectors, and wherein the fourth ions are directed by the second ion mirror to a second region of the second one or more ion detectors.

Embodiment 111. The parallel mass analyzer of embodiment 110, wherein the accelerator is configured to eject the first ions, the second ions, the third ions, and the fourth ions at about the same time.

Embodiment 112. The parallel mass analyzer of embodiment 110, wherein the accelerator is configured to eject the first ions and the third ions at a first time, and the second ions and fourth ions at a second time, wherein the first time and the second time are different.

Embodiment 113. The parallel mass analyzer of any one of embodiments 110-112, wherein the accelerator is configured to eject the first ions and the third ions in substantially opposite directions.

Embodiment 114. The parallel mass analyzer of any one of embodiments 110-113, wherein the accelerator is configured to eject the second ions and the fourth ions in substantially opposite directions.

Embodiment 115. The parallel mass analyzer of any one of embodiments 86-105, wherein the parallel mass analyzer is configured as the first mass analyzer and the second mass analyzer in the apparatus of any one of embodiments 1-44.

Embodiment 116. Use of the parallel mass analyzer of any one of embodiments 86-105 in the method of any one of embodiments 45-85, wherein the parallel mass analyzer is used as the first mass analyzer and the second mass analyzer.

Embodiment 117. A method for parallel mass analysis using the mass analyzer of any one of embodiments 86-115, the method comprising:
injecting the first ions into the flight tube using the accelerator;
injecting the second ions into the flight tube using the accelerator;
directing the first ions to the first region of the one or more ion detectors using the ion mirror;
directing the second ions to the second region of the one or more ion detectors using the ion mirror;
determining m/z for at least a portion of the first ions; and
determining m/z for at least a portion of the second ions.

Embodiment 118. A method of identifying analytes comprising:
obtaining data representing signal intensity against m/z for a plurality of overlapping mass windows;
comparing the overlapping mass windows to associate peaks with a narrower mass range that overlaps between two or more mass windows; and
identifying one or more analytes based on the narrower mass range and the peaks associated with the narrower mass range.

Embodiment 119. The method of embodiment 118, wherein the identifying one or more analytes comprises comparing a library of analyte mass data with the narrower mass range and the peaks associated with the narrower mass range.

Embodiment 120. The method of embodiment 119, wherein a probability of detection for an analyte within the library of analyte mass data is determined based on the comparison.

Embodiment 121. The method of embodiment 120, wherein an analyte is identified if the probability of detection exceeds a threshold (e.g., a pre-determined threshold).

Embodiment 122. The method of any one of embodiments 118-121, wherein the overlapping mass windows overlap by about one half of the mass window width.

Embodiment 123. The method of any one of embodiments 118-122, wherein the data is obtained using any one of the methods of embodiments 45-85.

Embodiment 124. A computer readable medium comprising machine-executable code that, upon execution by the one or more computer processors, implements any one of the methods of embodiments 45-85.

Embodiment 125. A computer readable medium comprising machine-executable code that, upon execution by the one or more computer processors, implements any one of the methods of embodiments 118-123.

What is claimed is:

1. An apparatus for mass spectrometry, the apparatus comprising:
an ion source;
an ion splitter, the ion splitter configured to receive an ion flux from the ion source and split the ion flux between two or more ion beam paths, wherein:
(i) the ion splitter comprises a split ion funnel; or
(ii) ions are transmitted through the ion splitter to the two or more ion beam paths at substantially the same time;
a first mass analyzer disposed along a first of the two or more ion beam paths; and
a second mass analyzer disposed along a second of the two or more ion beam paths,
wherein the ion splitter is disposed between (i) the ion source and (ii) the first mass analyzer and the second mass analyzer, and
wherein the first mass analyzer and the second mass analyzer are each configured to receive a portion of the ion flux from the ion splitter.

2. The apparatus of claim 1, wherein the ion splitter comprises a split ion funnel and ions are transmitted through the splitter to the two or more ion beam paths at substantially the same time.

3. The apparatus of claim 1, wherein the ion splitter is configured to split the ion flux between the two or more ion beam paths without introduction of a mass-to-charge or an ion-mobility bias between the two or more ion beam paths.

4. The apparatus of claim 3, wherein the ion splitter comprises a split ion funnel.

5. The apparatus of claim 4, wherein the split ion funnel evenly divides the ion flux between two or more ion paths.

6. The apparatus of claim 1, wherein a first ion path of the two or more ion paths comprises a first collision cell.

7. The apparatus of claim 6, wherein a second ion path of the two or more ion paths comprises a second collision cell.

8. The apparatus of claim 7, wherein the two or more mass analyzers comprise a time-of-flight mass analyzer.

9. The apparatus of claim 8, wherein the two or more mass analyzers comprise at least two time-of-flight mass analyzers.

10. The apparatus of claim 8, wherein the apparatus for parallel mass spectrometry is configured to detect a biomolecule from a biological sample.

11. The apparatus of claim 10, wherein the biomolecule is a protein.

12. The apparatus of claim 10, wherein the apparatus is configured to detect a plurality of biomolecules from the biological sample.

13. The apparatus of claim 1, further comprising a first ion mobility device.

14. The apparatus of claim 13, further comprising a second ion mobility device.

15. The apparatus of claim 13, wherein at least one of the first or second ion mobility devices is a field-asymmetric ion mobility spectrometry (FAIMS) device.

16. The apparatus of claim 14, wherein at least one of the first or second ion mobility devices is a travelling-wave ion mobility spectrometry (TWIMS) device.

17. The apparatus of claim 14, wherein the first or second ion mobility device is configured to direct ions having a first mobility to a first of the two or more ion beam paths and ions having a second ion mobility to a second of the two or more ion beam paths.

18. The apparatus of claim 1, wherein the ion source is an electrospray ion source.

19. The apparatus of claim 18, wherein the electrospray ion source comprises a nano-electrospray emitter.

20. The apparatus of claim 18, wherein the electrospray ion source comprises a plurality of nano-electrospray emitters.
